(19) United States
(12) Patent Application Publication
(10) Pub. No.: US 2023/0263388 A1
(43) Pub. Date: Aug. 24, 2023

(54) EYE EXAMINATION DEVICE, SYSTEM AND METHOD

(71) Applicant: Ashwani MADKAN, Tacoma, WA (US)

(72) Inventor: Ashwani MADKAN, Tacoma, WA (US)

(21) Appl. No.: 18/003,857

(22) PCT Filed: Jun. 30, 2021
(86) PCT No.: PCT/US2021/040015

§ 371 Date: Dec. 29, 2022

Related U.S. Application Data
(60) Provisional application No. 63/046,039, filed on Jun. 30, 2020.

(51) Int. Cl. A61B 3/04; A61B 3/00; A61B 3/103; G16H 40/67

(52) U.S. Cl. CPC A61B 3/04; A61B 3/0033; A61B 3/103; G16H 40/67

(57) ABSTRACT

A system includes an optical examination device and an eye-examination provider device remote from the optical examination device. The optical examination device includes a frame, which, in operation, positions the optical examination device with respect to a eyes of a patient. A plurality of sensors are embedded in the frame and sense data related to the eyes of the patient. Processing circuitry also is embedded in the frame and coupled to the plurality of sensors. A communication link is established between the optical examination device and the eye-examination provider device. The processing circuitry of the optical examination device executes optical examination routines on the patient using the plurality of sensors. The processing circuitry controls the optical examination routines using control signals received from the remote eye-examination provider device via the communication network.

### BACKGROUND

### Technical Field

The present disclosure generally relates to eye examination devices, systems and methods.

### Description of the Related Art

Due to increased awareness of health monitoring and early detection of health conditions individuals are often required to make visits to clinics for a range of tests and check-ups. One area in which there is a particular need for regular visits to clinics is eye examination. These eye examinations can range from the monitoring of health of the individual's eyes, to detecting and diagnosing disorders, diseases and other changes in the patient's eyes and/or vision. A typical routine eye examination process starts from scheduling appointments at the clinic, conducting on site eye examinations and visiting the clinic again to pick up a glasses frame or other prescriptions if any. When it comes to eye examinations at the clinic, scheduling appointments can be a time consuming task.

Current systems lack appropriate patient to provider ratios due to the localized nature of an optometrist's office. Unfortunately, for some users it is challenging due to longer waiting times in the clinic's waiting room. Furthermore, after the eye examination, the available options for picking a glasses frame or other prescriptions by the examinee may be limited, as the clinic may have or be limited on the different frames available.

### BRIEF SUMMARY

In an embodiment, an eye-examination is performed virtually or remotely using a device provided to the patient or user, facilitating examinations without requiring a visit to an examination clinic. Thus, an examination may be arranged without the need to schedule an eye examination appointment, leave the house, and physically go to a clinic. In an embodiment, a virtual or remote eye examination may provide a series of tests, such as various vision tests and health tests related to the patient's eyes. In an embodiment, test-related data may be saved, e.g., by the clinic performing the testing or the device, for future purposes as a reference to the medical record pertaining to the patient's eye health.

In an embodiment, a system includes an optical examination device and an eye-examination provider device remote from the optical examination device. The optical examination device includes a frame, which, in operation, positions the optical examination device with respect to a eyes of a patient. A plurality of sensors are embedded in the frame and sense data related to the eyes of the patient. Processing circuitry also is embedded in the frame and coupled to the plurality of sensors. A communication link is established between the optical examination device and the eye-examination provider device. The processing circuitry of the optical examination device executes optical examination routines on the patient using the plurality of sensors. The processing circuitry controls the optical examination routines using control signals received from the remote eye-examination provider device via the communication network.

In an embodiment, an optical examination device comprises: a frame, which, in operation, positions the optical examination device with respect to a eyes of a patient; a plurality of sensors embedded in the frame, which, in operation sense data related to the eyes of the patient; a communication interface embedded in the frame; and processing circuitry embedded in the frame and coupled to the plurality of sensors and to the communication interface. The processing circuitry, in operation: establishes a communication link with a remote eye-examination provider device via the communication interface; and executes a first plurality of optical examination routines on the patient using the plurality of sensors, wherein the first plurality of optical examination routines are controlled using control signals received from the remote eye-examination provider device via the communication link.

In an embodiment, a method comprises: establishing a communication link between an optical examination device and a remote eye-examination provider device; and executing a first plurality of optical examination routines on a patient using a plurality of sensors of the optical examination device, wherein the first plurality of optical examination routines are controlled using control signals received from the remote eye-examination provider device via the communication link.

### BRIEF DESCRIPTION OF THE SEVERAL VIEWS OF THE DRAWINGS

Non-limiting and non-exhaustive embodiments are described with reference to the following drawings, wherein like labels refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. Moreover, some elements known to those of skill in the art have not been illustrated in the drawings for ease of illustration. One or more embodiments are described hereinafter with reference to the accompanying drawings in which:

FIG. 1 shows an overview of an embodiment of an environment that may be employed to facilitate providing virtual or remote eye-examination and related services.

FIG. 2 shows an embodiment of a computing system and environment that may be employed to facilitate providing virtual or remote eye-examination and related services.

FIG. 3 is a flow chart illustrating a method to perform a remote or virtual eye examination according to one or more embodiments.

FIG. 4 is a perspective view of a remote or virtual eye examination device according to one or more embodiments.

### DETAILED DESCRIPTION

In the following description, certain details are set forth in order to provide a thorough understanding of various embodiments of devices, systems, methods and articles. However, one of skill in the art will understand that other embodiments may be practiced without these details. In other instances, well-known structures and methods associated with, for example, mobile devices, etc., such as processing cores, memories, power supplies, buses, communication networks, etc., have not been shown or described in detail in some figures to avoid unnecessarily obscuring descriptions of the embodiments.

Unless the context requires otherwise, throughout the specification and claims which follow, the word “comprise” and variations thereof, such as “comprising,” and “comprises,” are to be construed in an open, inclusive sense, that is, as “including, but not limited to.”

Reference throughout this specification to “one embodiment,” or “an embodiment” means that a particular feature, structure or characteristic described in connection with the embodiment is included in at least one embodiment. Thus, the appearances of the phrases “in one embodiment,” or “in an embodiment” in various places throughout this specification are not necessarily referring to the same embodiment, or to all embodiments. Furthermore, the particular features, structures, or characteristics may be combined in any suitable manner in one or more embodiments to obtain further embodiments.

The headings are provided for convenience only, and do not interpret the scope or meaning of this disclosure.

The sizes and relative positions of elements in the drawings are not necessarily drawn to scale. For example, the shapes of various elements and angles are not drawn to scale, and some of these elements are enlarged and positioned to improve drawing legibility. Further, the particular shapes of the elements as drawn are not necessarily intended to convey any information regarding the actual shape of particular elements, and have been selected solely for ease of recognition in the drawings.

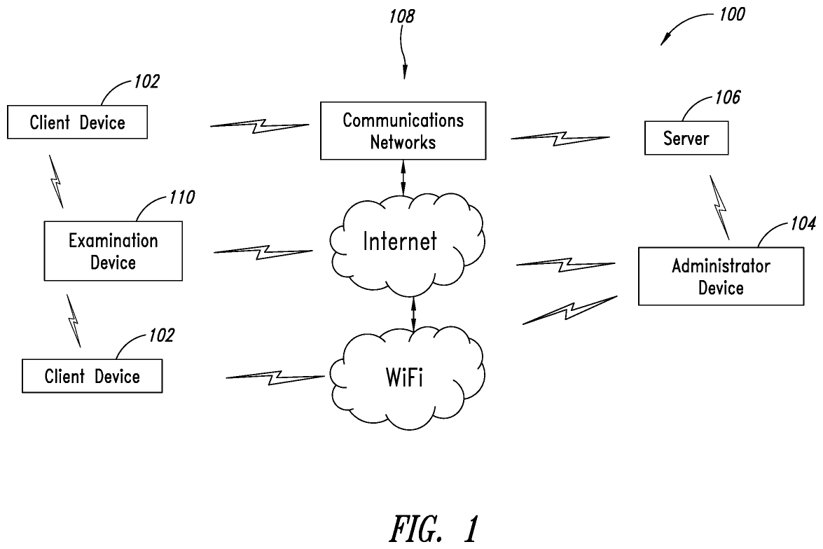

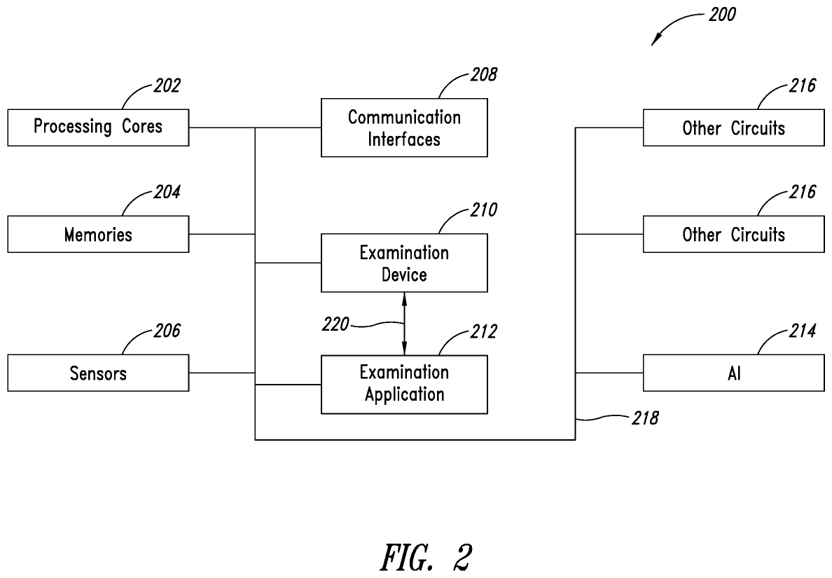

FIG. 1 shows an embodiment of an environment 100 that may be employed to facilitate providing eye-examination and related services as described herein, such as by performing one or more of the methods disclosed herein. The environment 100 includes one or more client devices 102 (e.g., smart phones, laptops, desktop computers), one or more administrator devices 104 (e.g., desktops computers), one or more servers 106, which may typically be remote from the client devices 102, and one or more eye examination devices 110. The devices 102, 104, 110 and the servers 106 may communicate over one or more communication networks 108. A provider of eye examination and related services may use one or more servers 106 in communication with an administrator device 104 to install and/or configure eye examination application or app installed on a client device 102 or an examination device 110. The devices 102, 104, 110, servers 106 and communication networks 108 may be configured to communicate with each other using various wired and wireless communication links. The client devices 102, administrator devices 104, eye examination devices 110 and servers 106 may be implemented, for example, using one or more of the environments 200 of FIG. 2.

FIG. 2 shows an embodiment of an environment or system 200 that may be employed to facilitate eye examination and related services as described herein, such as by performing one or more methods disclosed herein. The system 200 can be utilized to implement a client device, an examination device, a communication network, a server, an administrative device, etc., for example as described in relation to FIG. 1. The system 200 comprises one or more processing cores or circuits 202. The processing cores 202 may comprise, for example, one or more processors, a state machine, a microprocessor, a programmable logic circuit, discrete circuitry, logic gates, registers, etc., and various combinations thereof. The processing cores may control overall operation of the system 200, execution of application programs by the system 200, etc.

The system 200 includes one or more memories 204, such as one or more volatile and/or non-volatile memories which may store, for example, all or part of instructions and data related to control of the system 200, applications and operations performed by the system 200, etc. The one or more memories 204 may include one or more cache memories, one or more primary memories, one or more secondary memories, etc., each of which may comprise memory management circuitry. The one or more memories 204, in operation, may be shared by one or more processes executed by the system 200.

The system 200 may include one or more sensors 206 (e.g., image sensor, distance sensors, accelerometers, pressure sensors, temperature sensors, etc.), one or more communication interfaces 208 (e.g., wireless communication interfaces, wired communication interfaces, etc.), one or more artificial intelligence circuits or modules 214, for example, to implement a convolutional neural network (CNN), and one or more other circuits 216, which may include other functional circuits, antennas, power supplies, etc., and a main bus system 218. The system 200 also includes an examination device 210 and an examination application 212, which may, in operation, be employed to provide eye examination and related services such as described herein. The main bus system 218 may include one or more data, address, power and/or control buses coupled to the various components of the system 200. The system 200 also may include additional bus systems such as bus system 220, which communicatively couples the examination device 210 and the examination application 212.

Embodiments of the computing system 200 of FIG. 2 may not include all of the illustrated components of the computing system 200, may contain additional components not shown in FIG. 2, and may not be configured as shown in FIG. 2. For example, a computing system 200 configured as administrative device, server or client device may or may not include an examination device 210.

According to one embodiment, a remote eye examination device 110, 210 is provided for online eye examination of a user. The remote eye examination device is configured to perform an eye examination without the need to schedule an eye examination appointment, leave the house, and physically go to the clinic. The remote eye examination device is configured to be accessible by a remote eye exam provider, who can manipulate or control the device to utilize it. With reference to FIGS. 1 and 2, several different operational configurations may be employed to provide eye examination and related services.

For example, a patient may be provided with an examination device 110, and a provider may control operation of the examination device using an administrator device 104. The examination device 110 may be coupled to the administrative device directly through a communication network 108 (e.g., the Internet, a WAN, a LAN, a WiFi, and various combinations thereof). In some embodiments, the examination device 110 may be coupled to the communication network via a client device 102. Software and other control modules or circuits may be distributed in various manners among the various devices. For example, control circuitry and modules may be distributed between an examination device 110 and an administrator device 104 in some embodiments, and may be distributed between the examination device 110, the administrator device 104 and a client device 102 in other embodiments.

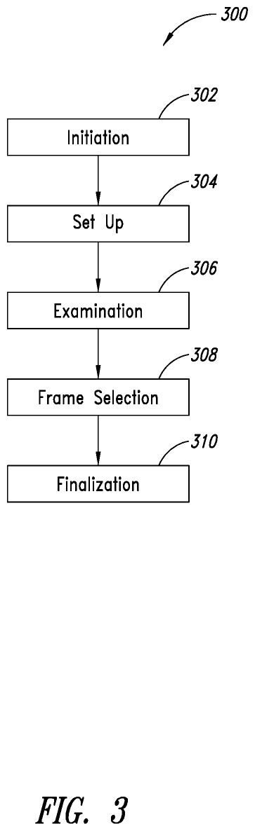

According to an embodiment, a method 300 for remote eye examination includes stages of an initiation 302 of remote eye examination, a set up of an eye examination device 304, performing an eye examination 306, selection of corrective eyewear 308 and finalization 310. The method facilitates allowing the user to perform an eye examination that will provide a series of tests, such as an eye wellness examination and a refraction prescription. The method 300 of FIG. 3 will be described for convenience with reference to FIGS. 1 and 2.

The remote eye examination device 110 is designed to be associated with a computer program product, such as examination application 212, to facilitate easy communication between a patient and a provider, and between the provider and the examination device 110, 210. The computer program product may include a code segment for generating one or more graphical user interfaces (“GUI”) for the patient and the provider. The GUIs can also be embodied in a computer program stored on computer readable media. The computer program may include code segments or routines to enable all of the functional aspects of the interface described or shown herein. The computer program may save data that is related to the exam, eye, or other aspects of the event to be used for the current examination or future examinations, for example, in secure cloud storage, or local storage (e.g., on an administrator or client device, for example, if the patient has any security concerns). The remote eye examination device may be user-specific, or may be fully transferable to different patients or providers, while retaining prior data if desired, e.g., so a patient can switch providers without the worry of losing information.

At 302, the method 300 initiates delivery of an eye examination device 110. For example, a patient may obtain an eye examination device 110, for example by ordering a device from a provider. A patient may request an eye examination device using a computer program product installed on a mobile device or a web portal. The patient also may call a customer center setup to place an order for the eye examination device. The patient may also visit locations where a device is available immediately, such as a pharmacy or rural town center. Once the eye examination device is received the patient will be able to start the exam by simply wearing the device. The patient can take the examination at a desired location (e.g., home, work, etc.). The method 300 proceeds from 302 to 304.

At 304, the method 300 set ups the examination device 110 to perform examinations and provide related services. During the set-up stage, the user or patient may activate an eye examination device 110. In some embodiments, the activation can be performed by simply opening the eye examination device 110. Once activated, a pre-examination staff member may get notified to login and facilitate the setup and working of the device. The pre-examination staff will also be able to communicate with the user or patient once the device is activated. After successful validation, the user or patient may be placed into a queue for the examination or an examination may be scheduled. During the course of the examination, as described below, pictures of the retina may be obtained that will allow the user to seamlessly login in the future by unique identification based on the images of the patient's retina. Other biometric information may be obtained in addition to or instead of retina images.

Once the staff member validates the operations, a user or patient may be allowed to request a provider at their convenience. This can be done immediately or scheduled at a later point when the user is available. The scheduling of the appointment can be completed through a corresponding mobile device application or through a call that allows the user to share their personal information such as address, any existing health problems, and any special considerations for the provider to know for the upcoming examination, billing information, etc. The appointment scheduling is so that the provider knows what time to perform the eye examination virtually with the user.

For example, a patient may download an application from a server 106 or administrative device 104 to the examination device 110 or a client device 102, and the application may configure the examination device 110 to provide desired eye examination and related services. As discussed above, the configuration may include receiving input data from the patient (e.g., identification information (name, biometric information), measurement data, medical history, etc.), control information from the provider (e.g., from an application executing on an administrator device 104) billing information; etc., and various combinations thereof. The set up procedure may include scheduling of an appointment for the eye examination, which may be a remote or virtual appointment. The method 300 proceeds from 304 to 306.

At 306, a remote or virtual eye examination is performed. The patient puts on or wears the examination device 110. A provider controls cameras and other sensors 206 on the examination device 110 via an administrator device 104. For example, a provider may control the examination device 110 to perform a series of tests to check on a patient's eyes. The eye examination stage 306 may be initiated after the set-up stage 304. During the eye examination stage 306, the provider may conduct a remote eye examination to perform the examination virtually using the examination device 110, 210. The examination may utilize one or more cameras and sensors on the eye examination device 110, 210, and one or more displays of the eye examination device 110, 210, operated under control of the system 200, for example, under control of an application executing on a system 200, such as an examination device 110, a client device 102, an administrator device 104, or various combinations thereof. The provider may control the use of cameras and sensors (e.g., to control movements of the cameras and sensors (e.g., positions in positioning tracks, pivot angles), sampling rates and timing, etc.) to complete a thorough examination and fully explore any oddities. All facets of the eye's shape, color, and overall image may be utilized by data algorithms (e.g., neural network classification systems) to determine if there are any anomalies ahead of actual patient presentation of symptoms to improve health outcomes for patients. It is noted that the movements may include stored movement patterns (e.g., a particular combination of positions in the movement tracks and pivot angles), and manually controlled movements (e.g., by the examiner using the administrator device).

A data algorithm, such as a trained neural network, may be employed to utilize exam data and changes in metrics taken during the course of the examination, for example to identify abnormal patterns or patterns associated with various conditions. In addition to the detection, the computer program may also be configured to predict models and utilize machine learning mechanisms to compare the user's growth patterns to known and unknown abnormal patterns and find new methods of identification of adverse health outcomes. The remote eye examination may include execution of a series of examinations that contain health tests of the eye along with vision tests. The health tests that the eye examination device may be designed to perform and detect blood vessel obstruction, cataracts, corneal injury, eye puff (non-contact tonometer), glaucoma screening, macular degeneration, predictive model against shape of eye to predict health concerns in advance, retina detachment, retinal exam, slit lamp exam, etc. and various combinations thereof. During the vision test, the eye examination device may perform several detection algorithms including but not limited to a color vision test, eye muscle, refraction assessment, visual acuity test, visual fields, etc., and various combinations thereof.

The method 300 proceeds from 306 to 308, where an optional frame selection process may be performed in accordance with some embodiments. The frame selection stage 308 of the remote eye examination may be initiated after the eye examination stage 306. The frame selection process 308 allows a patient to view themselves wearing various frames virtually. Several approaches, such as (i) a digital display of the outside of the frames on the examination device 110, 210, (ii) an augmented reality display with photos taken of the user presented either on a display (projector) of the examination device 110, 210 or on a display of a client device 102, (iii) static images with the frames overlaid on the patient on a display of the examination device 110, 210 or on a display of a client device 102, and various combinations thereof, may be employed. The patient will have access to switching to different frames and viewing how they look with each different frame, without the need to physically have the frames available for swapping. Once the user or patient is satisfied and chooses a glasses frame, the user may place an order, for example, using an application executing on the examination device 110, 210 or on a client device 102. In some embodiments, the patient may be required to return the eye examination device 110, 210 to release the order of the prescription glasses. The user or patient also may have the ability to order contacts using the application in addition to or instead of glasses. Distances such as the pupillary distance, nose to ear, and ear to ear may additionally be captured to best custom fit the frames for the patient.

The augmented reality display can include a head-up display of the various frames for users overlaid on a real world view of the frames. The augmented reality content may comprise an indication of the types and shapes of the frames that are predicted to best fit on the patient such that the user is provided with an augmented reality view comprising the real world view of the frames.

The method 300 proceeds from 308 to 310. At 310, the glasses or contacts are delivered and an optional follow-up examination may be performed. The follow-up examination may include fit and prescription verification to ensure the correct quality of glasses frame and lens provided to the patient. In addition, data gather by sensors on the eye examination device 110, 210 may facilitate custom fitting of the frames the patient instead of mass produced frames providing a universal fit.

Embodiments of the method 300 of FIG. 3 may contain additional acts not shown in FIG. 3, may not contain all of the acts shown in FIG. 3, may perform acts shown in FIG. 3 in various orders, may combine acts, and may be modified in various respects. For example, the initiation and set-up steps may be combined in some embodiments (e.g., the examination device 110 may be picked up at a distribution center and the set up process performed as part of the process of receiving the examination device). In another example, in some embodiments the frame selection process 308 may be omitted or replaced with a contact ordering process.

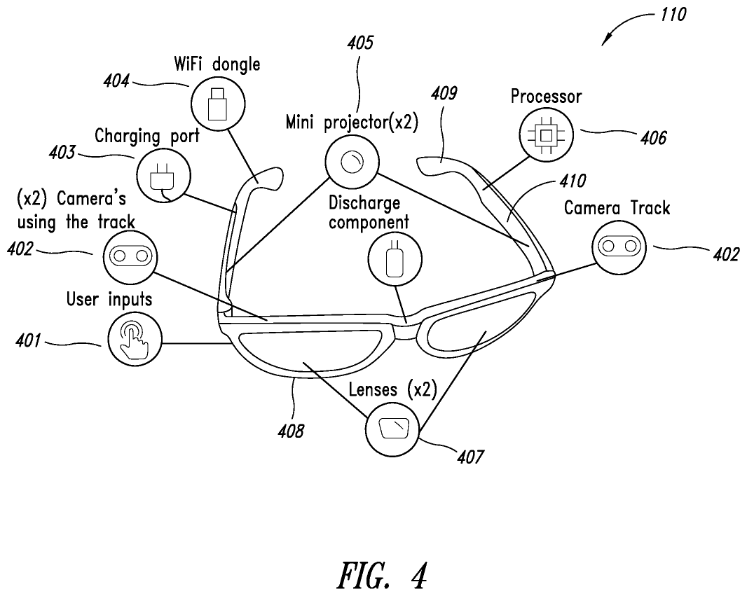

FIG. 4 is a perspective view of an embodiment of a eye examination device 110 according to one or more embodiments. As illustrated, the remote eye examination device 110 includes components to interact directly with the human eye to automate an eye examination process, such as the process described above with respect to FIG. 3. In addition to standard eyeglass elements, such as a frame 408, lenses 407, earpieces 409 and temples 410, the remote eye examination device 110 includes a input device 401 (e.g., a button, a microphone, etc), one or more cameras or other sensors 402 (e.g., image sensors, distance sensors, pressure sensors, etc.), a charging port or interface 403 (e.g., a USB port or a wireless charging interface), a communication interface 404 (e.g., NFC, WiFi, LTE, etc., and various combinations thereof), a projector 405 and a processor 406.

The frame and lenses will serve to allow the lens, cameras and sensors of the device to be placed in front of the user's eyes and provide an unobstructed viewing position directly in front of the user's eyes. The earpieces 409 may comprise one or more speakers to allow the user to hear any instructions from the device, whether automated or from a provider. The input device will allow the user to physically respond to any potential queues or examination prompts where direct user input would be desirable. The cameras 402 may operate both in the visible spectrum as well as outside the visible spectrum when appropriate, such as, when obtaining a focal point calculation for a patient. A communication interface 404 facilitates the device 110 communicating with the environment 100. In extremely rural areas, the communication interface 404 may include a satellite communication device. A projector 405 is also available to help produce images for the user to view and make appropriate responses (e.g., respond to examination inquiries regarding projected images, select frames, etc.).

The processor 406 interfaces with all user inputs, cameras, and any additional sensors embedded on the headset via a data bus. Information may be aggregated and passed back to processor 406 via a data bus connector, or wireless transmission. The process 406 may interface with servers (see administrator device 104 and server 106 of FIG. 1), websites, and or graphical user interfaces, storing and relaying information during examination and for later review.

The remote eye examination device 110 is configured to perform several components of a comprehensive eye examination including but not limited to a visual field test, color vision test, ocular motility test, refraction assessment, and visual acuity test. The typical refraction assessment may also include a refraction examination. The vision field test can be broken down into two parts. The first part of the visual field test is confrontation where one eye is covered at a time to evaluate on the visual field of an individual eye. The second part may be achieved by determining the user's extent of visual field and may be accomplished by automated eye-tracking of the user or by an interactive algorithm in which the user presses a button when light is visible for the user so that the extent of visual field can be plotted.

The device 110 may also be configured to perform color vision testing (screening), for example, by displaying a couple of Ishihara plates on any digital display of reasonable color accuracy. During an eye muscle examination the device 110 may be designed to utilize eye tracking algorithms and will allow the user to follow a moving object/digital image. The system or provider may monitor whether the eye movement is 1) full or complete, 2) moves freely and 3) that the movement is painless. Ishihara plates are part of a widely used standard screening test for color blindness that consists of a set of plates covered with colored dots which the test subject views in order to find a number composed of dots of one color which a person with various defects of color vision will confuse with surrounding dots of color {based on the optical phenomenon of pseudo-isochromatism, where colors that appear alike (isochromatic) are in fact different (pseudo)}.

The refraction may accomplished using a computerized autorefractor (objective refraction) and phoropter (subjective refraction) to identify focus in the user's retina and determine the optical correction to see a clear image. The various methods may use light rays and a computer processing to measure the objective refraction parameters needed for the user's eye, sphere, cylinder and axis. Several cameras may be employed to facilitate this analysis and calculation including cameras/sensors in the infrared and other non-visible spectrums. The device 110 may be configured to confirm this refractive error (if any) by displaying optotypes (such as letters) to alter the users focal power to find the optimal vision correction by a phoroptor.

For a glaucoma screening and retinal exam, the device 110 may be configured to photograph the retina, the optic disc and the underlying layer of blood vessels that nourish the retina (choroid) and check for any damage. In addition, an optical microscope may be utilized whose purpose is to examine and capture images of both the external and internal anatomical structures of the eyeball.

In an embodiment, an optical examination device comprises a frame, which, in operation, positions the optical examination device with respect to a eyes of a patient, a plurality of sensors embedded in the frame, which, in operation sense data related to the eyes of the patient, a communication interface embedded in the frame, and processing circuitry embedded in the frame and coupled to the plurality of sensors and to the communication interface. The processing circuitry, in operation, establishes a communication link with a remote eye-examination provider device via the communication interface, and executes a first plurality of optical examination routines on the patient using the plurality of sensors, wherein the first plurality of optical examination routines are controlled using control signals received from the remote eye-examination provider device via the communication link. In an embodiment, the processing circuitry, in operation, initiates establishment of the communication link in response to an opening of the optical examination device or a receipt of user input. In an embodiment, the processing circuitry, in operation, sends patient identification information to the remote eye-examination provider device via the communication interface prior to the execution of the first plurality of optical examination routines. In an embodiment, the processing circuitry, in operation, waits for validation of the patient identification information by the remote eye-examination provider device before executing the first plurality of optical examination routines. In an embodiment, the first plurality of optical examination routines include a color vision test, an eye muscle test, a refraction assessment test, a visual acuity test, a visual fields test, or various combinations thereof. In an embodiment, the optical examination device comprises projection circuitry embedded in the frame, and the processing circuitry, in operation, controls generating of an augmented reality display by the projection circuitry. In an embodiment, the augmented reality display includes a head-up display of frames for glasses overlaid on a real world view of the frames, wherein the augmented reality content comprises an indication of types and shapes of frames predicted to bestfit on the patient. In an embodiment, the processing circuitry, in operation, executes a second plurality of optical examination routines on the patient using the plurality of sensors, wherein the second plurality of optical examination routines are controlled using control signals received from remote eye-examination provider device. In an embodiment, the second plurality of optical examination routines verify a fit of a pair of eyeglasses for the patient. In an embodiment, the second plurality of optical examination routines verify a prescription of a pair of eyeglasses prescribed for the patient based on results of the first plurality of optical examination routines.

In an embodiment, a system includes an optical examination device and an eye-examination provider device remote from the optical examination device. The optical examination device includes a frame, which, in operation, positions the optical examination device with respect to a eyes of a patient. A plurality of sensors are embedded in the frame and sense data related to the eyes of the patient. Processing circuitry also is embedded in the frame and coupled to the plurality of sensors. A communication link is established between the optical examination device and the eye-examination provider device. The processing circuitry of the optical examination device executes optical examination routines on the patient using the plurality of sensors. The processing circuitry controls the optical examination routines using control signals received from the remote eye-examination provider device via the communication network. In an embodiment, the processing circuitry, in operation, initiates establishment of the communication link in response to an opening of the optical examination device or a receipt of user input. In an embodiment, the first plurality of optical examination routines include a color vision test, an eye muscle test, a refraction assessment test, a visual acuity test, a visual fields test, or various combinations thereof. In an embodiment, the optical examination device comprises projection circuitry embedded in the frame, wherein the processing circuitry, in operation, controls generating of an augmented reality display by the projection circuitry.

In an embodiment, a method comprises establishing a communication link between an optical examination device and a remote eye-examination provider device, and executing a first plurality of optical examination routines on a patient using a plurality of sensors of the optical examination device, wherein the first plurality of optical examination routines are controlled using control signals received from the remote eye-examination provider device via the communication link. In an embodiment, the method comprises initiating establishment of the communication link in response to an opening of the optical examination device or a receipt of user input. In an embodiment, the method comprises sending patient identification information to the remote eye-examination provider device via the communication interface and waiting for validation of the patient identification information prior to executing the first plurality of optical examination routines. In an embodiment, the first plurality of optical examination routines include a color vision test, an eye muscle test, a refraction assessment test, a visual acuity test, a visual fields test, or various combinations thereof. In an embodiment, the method comprises generating of an augmented reality display using projection circuitry of the optical examination device. In an embodiment, the method comprises providing a pair of prescription glasses to the patient based on results of the first plurality of optical examination routines, and executing a second plurality of optical examination routines using the optical examination device, wherein the second plurality of optical examination routines verify a fit of the pair of eyeglasses and a prescription of the pair of eyeglasses. In an embodiment, a non-transitory computer-readable medium's contents configure a processing device to perform an embodiment of a method disclosed herein.

Some embodiments may take the form of or comprise computer program products. For example, according to one embodiment there is provided a computer readable medium comprising a computer program adapted to perform one or more of the methods or functions described above. The medium may be a physical storage medium, such as for example a ROM chip, or a disk such as a Digital Versatile Disk (DVD-ROM), Compact Disk (CD-ROM), a hard disk, a memory, a network, or a portable media article to be read by an appropriate drive or via an appropriate connection, including as encoded in one or more barcodes or other related codes stored on one or more such computer-readable mediums and being readable by an appropriate reader device.

Furthermore, in some embodiments, some or all of the methods and/or functionality may be implemented or provided in other manners, such as at least partially in firmware and/or hardware, including, but not limited to, one or more application-specific integrated circuits (ASICs), digital signal processors, discrete circuitry, logic gates, standard integrated circuits, controllers (e.g., by executing appropriate instructions, and including microcontrollers and/or embedded controllers), field-programmable gate arrays (FPGAs), complex programmable logic devices (CPLDs), etc., as well as devices that employ RFID technology, and various combinations thereof.

The various embodiments described above can be combined to provide further embodiments. Aspects of the embodiments can be modified, if necessary to employ concepts of the various patents, applications and publications to provide yet further embodiments.

This application claims the benefit of priority to U.S. Provisional Application No. 63/046,039, filed Jun. 30, 2020, which application is hereby incorporated by reference in its entirety.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. An optical examination device, comprising:
a frame, which, in operation, positions the optical examination device with respect to a eyes of a patient;
a plurality of sensors embedded in the frame, which, in operation sense data related to the eyes of the patient;
a communication interface embedded in the frame; and
processing circuitry embedded in the frame and coupled to the plurality of sensors and to the communication interface, wherein the processing circuitry, in operation:
establishes a communication link with a remote eye-examination provider device via the communication interface; and
executes a first plurality of optical examination routines on the patient using the plurality of sensors, wherein the first plurality of optical examination routines are controlled using control signals received from the remote eye-examination provider device via the communication link.

2. The optical examination device of claim 1, wherein the processing circuitry, in operation, initiates establishment of the communication link in response to an opening of the optical examination device or a receipt of user input.

3. The optical examination device of claim 1, wherein the processing circuitry, in operation, sends patient identification information to the remote eye-examination provider device via the communication interface prior to the execution of the first plurality of optical examination routines.

4. The optical examination device of claim 3, wherein the processing circuitry, in operation, waits for validation of the patient identification information by the remote eye-examination provider device before executing the first plurality of optical examination routines.

5. The optical examination device of claim 1, wherein the first plurality of optical examination routines include a color vision test, an eye muscle test, a refraction assessment test, a visual acuity test, a visual fields test, or various combinations thereof

6. The optical examination device of claim 1, comprising projection circuitry embedded in the frame, wherein the processing circuitry, in operation, controls generating of an augmented reality display by the projection circuitry.

7. The optical examination device of claim 6, wherein the augmented reality display includes a head-up display of frames for glasses overlaid on a real world view of the frames, wherein the augmented reality content comprises an indication of types and shapes of frames predicted to bestfit on the patient.

8. The optical examination device of claim 1, wherein the processing circuitry, in operation, executes a second plurality of optical examination routines on the patient using the plurality of sensors, wherein the second plurality of optical examination routines are controlled using control signals received from remote eye-examination provider device.

9. The optical examination device of claim 8, wherein the second plurality of optical examination routines verify a fit of a pair of eyeglasses for the patient.

10. The optical examination device of claim 8, wherein the second plurality of optical examination routines verify a prescription of a pair of eyeglasses prescribed for the patient based on results of the first plurality of optical examination routines.

11. A system, comprising:
an optical examination device, including:
a frame, which, in operation, positions the optical examination device with respect to a eyes of a patient;
a plurality of sensors embedded in the frame, which, in operation sense data related to the eyes of the patient; and
processing circuitry embedded in the frame and coupled to the plurality of sensors;
an eye-examination provider device remote from the optical examination device; and
a communication network coupled to the optical examination device and to the eye-examination provider device, wherein the processing circuitry of the optical examination device, in operation:
establishes a communication link with the remote eye-examination provider device via the communication network; and
executes a first plurality of optical examination routines on the patient using the plurality of sensors, wherein the first plurality of optical examination routines are controlled using control signals received from the remote eye-examination provider device via the communication network.

12. The system of claim 11, wherein the processing circuitry, in operation, initiates establishment of the communication link in response to an opening of the optical examination device or a receipt of user input.

13. The system of claim 11, wherein the first plurality of optical examination routines include a color vision test, an eye muscle test, a refraction assessment test, a visual acuity test, a visual fields test, or various combinations thereof.

14. The system of claim 11, wherein the optical examination device comprises projection circuitry embedded in the frame, wherein the processing circuitry, in operation, controls generating of an augmented reality display by the projection circuitry.

15. A method, comprising:
establishing a communication link between an optical examination device and a remote eye-examination provider device; and
executing a first plurality of optical examination routines on a patient using a plurality of sensors of the optical examination device, wherein the first plurality of optical examination routines are controlled using control signals received from the remote eye-examination provider device via the communication link.

16. The method of claim 15, comprising initiating establishment of the communication link in response to an opening of the optical examination device or a receipt of user input.

17. The method of claim 15, comprising sending patient identification information to the remote eye-examination provider device via the communication interface and waiting for validation of the patient identification information prior to executing the first plurality of optical examination routines.

18. The method of claim 15, wherein the first plurality of optical examination routines include a color vision test, an eye muscle test, a refraction assessment test, a visual acuity test, a visual fields test, or various combinations thereof.

19. The method of claim 15, comprising generating of an augmented reality display using projection circuitry of the optical examination device.

20. The method of claim 15, comprising:
providing a pair of prescription glasses to the patient based on results of the first plurality of optical examination routines; and
executing a second plurality of optical examination routines using the optical examination device, wherein the second plurality of optical examination routines verify a fit of the pair of eyeglasses and a prescription of the pair of eyeglasses.
